(19) United States
(12) Patent Application Publication
(10) Pub. No.: US 2008/0306288 A1
(43) Pub. Date: Dec. 11, 2008

(54) Method for Producing Olefin Oxides and Peroxides, Reactor and the Use Thereof

(75) Inventors: Steffen Schirrmeister, Muehlheim An Der Ruhr (DE); Bernd Langanke, Holzwickede (DE); Karsten Bueker, Dortmund (DE); Frank Becker, Offenbach (DE); Johannes Albrecht, Woellstadt (DE); Georg Markowz, Alzenau (DE); Ruediger Schutte, Alzenau (DE)

Correspondence Address: CONNOLLY BOVE LODGE & HUTZ, LLP, P O BOX 2207, WILMINGTON, DE, 19899, US

(73) Assignee: UHDE GMBH, Dortmund (DE)

(21) Appl. No.: 11/665,357

(22) PCT Filed: Sep. 16, 2005
(86) PCT No.: PCT/EP05/09965

§ 371 Date: Apr. 29, 2008

(30) Foreign Application Priority Data
Oct. 15, 2004 (DE) 10 2004 050 506.3

(51) Int. Cl. C07D 301/02; C07C 409/00

(52) U.S. Cl. 549/518; 568/300

(57) ABSTRACT

A process for reactions with or to form peroxidic compounds in a wall reactor whose reaction space has a specific material coating is described.

Both higher space-time yields and increased selectivities can be achieved by means of the process.

The present invention relates to a process for preparing olefin oxides, in particular propene oxide, and also peroxides by heterogeneously catalyzed gas-phase oxidation in a wall reactor and also to the use of particularly suitable reactors in the gas-phase oxidation.

The epoxidation of olefins such as propene using oxygen in the liquid phase or in the gas phase is known.

DE 19748481 A1 describes a static micromixer and also a microreactor having a specific microgeometry and also their use for preparing oxiranes in the gas phase by catalytic oxidation of unsaturated compounds by means of air or by means of oxygen.

The epoxidation of olefins such as propene using hydrogen peroxide in the liquid phase or in the gas phase is a relatively new process variant.

Thus, U.S. Pat. No. 5,874,596 and DE-A-197 31 627 describe the epoxidation of olefins in the liquid phase using a titanium silicalite catalyst. A disadvantage of this process is the rapid deactivation of the catalyst by high-boiling by-products.

The use of a wall reactor, more precisely a microreator, in the oxidation of organic compounds in the liquid phase is known from EP-A-903,174. Here, a cooled microreactor in which the heat produced by the exothermic oxidation reaction with peroxides can be removed more rapidly is used. The decomposition of the liquid peroxide compound can be kept low by carrying out the reaction at moderate temperatures.

U.S. Pat. No. 4,374,260 discloses the epoxidation of ethylene in the gas phase using a silver-containing catalyst at from 200 to 300° C. Epoxidizing agents used are air or molecular oxygen.

Further epoxidation reactions of reactants in the gas phase are known from U.S. Pat. No. 5,618,954 in which 3,4-epoxy-1-butene is reacted over a silver-containing catalyst by means of oxygen-containing gases in the presence of water in a fixed-bed reactor at temperatures of from 100 to 400° C.

Attempts have also already been made to epoxidize lower olefins by means of hydrogen peroxide in the gas phase, with hydrogen peroxide being activated thermally or catalytically (cf. G. M. Mamedjarov and T. M. Nagiev, in Azerb. Khim. Zh. (1981), 57-60, and T. M. Nagiev et al. in Neftekhimiya 31 (1991), 670-675). A disadvantage is the high reaction temperatures which stand in the way of an economical process.

A further process uses an Si-containing catalyst and reaction temperatures of from 425 to 500° C. (cf. H. M. Gusenov et al. in Azerb. Khim. Zh. (1984), 47-51). Here, a tube reactor is used and the propene conversion is in the range from 15 to 65%.

Another process uses an Fe-containing catalyst (cf. T. M. Nagiev et al. in Neftekhimiya 31 (1991), 670-675). The reaction yields are about 30% and the catalyst has a very short operating life. Longer operating lives and a further reduction in the reaction temperature can be achieved using an Fe^{III}OH-protoporphyrin catalyst bound to aluminum oxide as support. When this catalyst is used, a propene oxide yield of about 50% is obtained at a temperature of 160° C. and a molar feed ratio of C₃H₆:H₂O₂:H₂O=1:0.2:0.8.

An improved process for the epoxidation of C₂-C₆-olefins in the gas phase is described in DE-A-100 02 514. The reaction is carried out using gaseous hydrogen peroxide in the presence of selected catalysts. Fixed-bed and fluidized-bed reactors are mentioned as suitable reactors. According to this document, the reaction is carried out at temperatures below 250° C., preferably in the range from 60 to 150° C., and the olefin is used in equimolar amounts, preferably in excess.

Carrying out the gas-phase epoxidation of propene using H₂O₂ in a wall reactor, more precisely a microreator, is known. For example, Kruppa and Schüth have examined the epoxidation reaction in, inter alia, a microreactor (IMRET 7,2003).

In Chemie Ingenieur Technik 2004, 76(5), 620-5, G. Markowz et al. describe the gas-phase epoxidation of propene to propene oxide using gaseous hydrogen peroxide over titanium silicalite catalysts in a microreactor. Details regarding the reactor design and technical reaction conditions are not disclosed.

Proceeding from this prior art, it is an object of the present invention to provide an improved process for the catalytic gas-phase epoxidation of olefins by means of peroxidic compounds, in which a high space-time yield combined with a high selectivity of the conversion of the thermally labile material of value to the product is achieved with a view to industrial use. Another object of the invention is an improved process for preparing peroxides.

It has surprisingly been found that when wall reactors which have a catalyst content and in which at least one dimension of the reaction space is kept below 1 cm and whose interior walls are coated with specific materials are used, the product selectivity of the peroxidic oxidant is, in contrast to classical fixed-bed reactors, increased when the reaction temperature is increased and higher selectivities of the peroxidic oxidant used are found as a result. Furthermore, it has been found that peroxidic compounds also surprisingly have increased stabilities in the special reactors, so that these reactors are also suitable for the synthesis of peroxidic compounds.

A further object of the present invention is to provide a reactor which is particularly suitable for the gas-phase reaction with and to form peroxidic compounds.

The present invention provides a process for preparing an olefin oxide by heterogeneously catalyzed gas-phase epoxidation of an olefin by means of a peroxidic compound in the presence of water and, if appropriate, an inert gas, which comprises the measures:
i) carrying out the gas-phase epoxidation at temperatures above 100° C.,
ii) use of a reactor which has at least one reaction space having at least one dimension of less than 10 mm,
iii) and in which the surface of the reaction space has a layer comprising aluminum oxide, zirconium oxide, tantalum oxide, silicon dioxide, tin oxide, glass and/or enamel and
iv) in which the reaction space contains catalyst, preferably is coated or partly coated with catalyst.

To carry out the process of the invention, it is possible to use all wall reactors or microreactors known per se. For the purposes of the present description, wall reactors are reactors in which at least one of the dimensions of the reaction space or the reaction spaces is less than 10 mm, preferably less than 1 mm, particularly preferably less than 0.5 mm.

The catalyst content of the reaction space/spaces can also be extended to collector or distributor spaces which can have a catalyst content different from the reaction space.

The reactor can have one reaction space or preferably a plurality of reaction spaces, more preferably a plurality of reaction spaces running parallel to one another.

The reaction spaces can have any dimensions, provided that at least one dimension is less than 10 mm.

The reaction spaces can have round, ellipsoidal, triangular or polygonal, in particular rectangular or square, cross sections. The or a dimension of the cross section is preferably less than 10 mm, i.e. at least one lateral dimension or the or a diameter.

In a particularly preferred embodiment, the cross section is rectangular or round and only one dimension of the cross section, i.e. a lateral dimension or the diameter, is less than 10 mm.

The reactor can be made of any material of construction as long as it is stable under the reaction conditions, allows satisfactory heat removal and the surface of the reaction space is completely or partly coated with the abovementioned specific materials.

Thus, the reactor can be made of metallic materials provided that the reaction space or reaction spaces is/are coated with aluminum oxide, zirconium oxide, tantalum oxide, silicon dioxide, tin oxide, glass and/or enamel.

Typical proportions of the sum of the oxides and/or glasses mentioned in the surface layer of the reaction space are in the range from 20 to 100% by weight, based on the material forming the surface layer of the reaction space.

In a particularly preferred embodiment, the reactor or at least the parts enclosing the reaction space comprise aluminum or an aluminum alloy. As is known, this material oxidizes in the presence of hydroperoxidic compounds to form aluminum oxide.

A further feature of the reactor used according to the invention is that all or part of the reaction space contains catalyst. Preference is given to the surface of the reaction space being partly or completely coated with catalyst.

The catalyst can be applied to the special surface of the substrate or the reaction space is entirely or partly filled with finely divided, supported or unsupported catalyst. The volume filled or coated with catalyst is porous and permeable to the reactants under the reaction conditions in the reactor, so that these, too, can come into contact with the specific materials.

It has surprisingly been found that when the specific materials mentioned are used under the reaction conditions the selectivity of the desired reaction increases with temperature and the product yield of the peroxide used or produced is increased thereby.

The present invention therefore also provides a process for preparing a peroxidic compound by means of a heterogeneously catalyzed gas-phase reaction, which comprises the measures:
v) carrying out the reaction by reaction of a precursor of the peroxidic compound with oxygen and/or an oxygen-containing compound to form the peroxidic compound at temperatures above 100° C.,
vi) use of a reactor which has at least one reaction space having at least one dimension of less than 10 mm,
vii) and in which the surface of the reaction space has a layer comprising aluminum oxide, zirconium oxide, tantalum oxide, silicon dioxide, tin oxide, glass and/or enamel and
viii) in which the reaction space may contain catalyst, preferably is coated or partly coated with catalyst.

The precursor of peroxidic compounds is generally oxygen. Thus, the invention encompasses the preparation of hydrogen peroxide from hydrogen and oxygen in a particular reactor. It is also possible to react organic molecules with hydrogen peroxide to form organoperoxidic compounds, e.g. peracetic acid.

The invention also provides a reactor for the reaction with or to form peroxidic compounds, which comprises:
a) at least one reaction space having at least one dimension of less than 10 mm,
b) the surface of the reaction space has a layer comprising aluminum oxide, zirconium oxide, tantalum oxide, silicon dioxide, tin oxide, glass and/or enamel and
c) the reaction space contains catalyst, with preference being given to the surface of the reaction space being coated or partly coated with catalyst.

The invention further provides for the use of the specially coated reactors in gas-phase oxidation by means of peroxidic compounds or in the synthesis of peroxidic compounds, in particular in heterogeneously catalyzed gas-phase reactions.

In a particularly preferred embodiment of the process of the invention, the gas-phase epoxidation is carried out in a microreactor which has a plurality of spaces which are arranged vertically or horizontally in parallel and each have at least one inlet and one outlet, with the spaces being formed by stacked plates or layers and part of the spaces representing reaction spaces having at least one dimension of less than 10 mm and the other part of the spaces representing heat transport spaces and the inlets into the reaction spaces being connected to at least two distributor units and the outlets from the reaction spaces being connected to at least one collector unit and the heat transport between reaction spaces and heat transport spaces occurring through at least one common wall which is formed by a common plate.

A particularly preferred microreactor of this type has spacer elements in all spaces, contains catalyst material applied to at least part of the interior walls of the reaction spaces, has a hydraulic diameter defined as the ratio of four times the area to the circumference of the free flow cross section in the reaction spaces of less than 4000 μm, preferably less than 1500 μm and particularly preferably less than 500 μm, and has a ratio of the vertically smallest distance between adjacent spacer elements to the slit height of the reaction space after coating with catalyst of less than 800 and greater than or equal to 10, preferably less than 450 and particularly preferably less than 100.

As olefins, it is possible to use all compounds which have one or more double bonds. Straight-chain or branched and also cyclic olefins can be used. The olefins can also be used as mixtures.

The olefinic starting materials have at least two carbon atoms. It is possible to use olefins having any number of carbon atoms, provided that they are sufficiently thermally stable under the conditions of the gas-phase epoxidation.

Preference is given to using olefins having from 2 to 6 carbon atoms. Examples are ethene, propene, 1-butene, 2-butene, isobutene and also pentenes and hexenes including cyclohexene and cyclopentene or mixtures of two or more of these olefins, but also higher olefins. The process is particularly useful for preparing propene oxide from propene.

As peroxidic compounds, it is possible to use H₂O₂, hydroperoxides or organic peroxides having any hydrocarbon radicals, provided that they are sufficiently thermally stable under the conditions of the gas-phase reaction.

As hydrogen peroxide, it is possible to use all vaporizable compositions comprising H₂O₂. It is advantageous to use aqueous solutions which contain from 30 to 90% by weight of hydrogen peroxide and are vaporized and fed to the wall reactor. The gaseous hydrogen peroxide is obtained by vaporization in an apparatus suitable for this purpose. To reduce subsequent reactions with the water coming from vaporization of aqueous hydrogen peroxide, preference is given to feeding highly concentrated H₂O₂ solutions to the vaporizer. The energy consumption is also reduced thereby.

As catalysts, it is possible to use any catalysts for the gas-phase oxidation of olefins by means of hydrogen peroxide.

One class of suitable and preferred catalysts is molecular sieves, in particular synthetic zeolites. A particularly preferred catalyst from the group consisting of molecular sieves is based on titanium-containing molecular sieves of the formula (SiO₂)₁₋ₓ(TiO₂)ₓ, e.g. titanium silicalite-1 (TS1) having an MFI crystal structure, titanium silicalite-2 (TS-2) having an MEL crystal structure, titanium beta-zeolite having a BEA crystal structure and titanium silicalite-48 having the crystal structure of zeolite ZSM 48. The TiO₂ content of TS-1 is preferably in the range from 2 to 4%. Titanium silicalites are commercially available. Instead of pure titanium silicalites, it is also possible to use combination products which comprise amorphous or crystalline oxides such as SiO₂, TiO₂, Al₂O₃ and/or ZrO₂ in addition to titanium silicalite.

Here, crystallites of titanium silicalite can be homogeneously distributed among the crystallites of the other oxides and form granules or be located as an outer shell on a core of other oxides.

Another class is metal-organic catalysts, for example iron-organic (protoporphyrin) or titanium-organic compounds on a suitable support.

A further class of preferred catalysts is preferably inorganic, in particular oxidic compounds which contain one or more elements of transition groups 4 to 6 of the Periodic Table and/or an arsenic and/or selenium compound as catalytically active element.

Particular preference is given to compounds of titanium, vanadium, chromium, molybdenum and tungsten.

The catalytic action of these compounds is considered to be, without ruling out other mechanisms, activation of the peroxidic starting material by the porous structure of the catalyst and/or by the ability of the catalyst to form peroxo compounds reversibly.

Particularly suitable catalysts are vanadium oxides, vanadates and their H₂O₂ adducts.

A further particularly suitable class of epoxidation catalysts comprises molybdenum or tungsten. Examples are MoO₃ and WO₃, molybdic and tungstic acids, alkali metal and alkaline earth metal molybdates and tungstates as long as their basicity does not lead to hydrolysis of the epoxide, homopolymolybdates, homopolytungstates, heteropolymolybdates and heteropolytungstates (=homopolyacids and heteropolyacids) and H₂O₂ adducts of the classes of substances mentioned, e.g. peroxomolybdic acid, peroxotungstic acid, peroxomolybdates and peroxotungstates, which can also be formed in situ from other Mo and W compounds during the epoxidation.

Catalysts for the preparation of hydrogen peroxide are, for example, gold, palladium or other noble metals on suitable supports, e.g. on carbons or on SiO₂. In general, no catalyst is required for the preparation of organo-peroxidic compounds.

To prepare a particularly suitable coating, the catalyst was applied together with a binder which is inert in respect of the epoxidation reaction to part of or all walls of the reaction space. A particular challenge is with regard to the very inert properties of the binder toward the gaseous peroxidic compound.

There are numerous examples of inactive binders for liquid applications. However, most substances display significant differences in their catalytic decomposition properties toward a gaseous peroxidic compound. The use of a coating comprising aluminum oxide, silicon dioxide or silicate has been found to be particularly preferred. These preferred catalytic coatings can be produced by mixing of the inactive binder with the active component, preferably with the pulverulent active component, shaping and heat treatment.

In another embodiment, catalysts whose active component has been applied to a porous support are used. In this way, it is possible to produce a particularly large internal volume which leads to particularly high reaction yields.

The starting materials for the process of the invention are fed into the wall reactor. The feed streams can contain further components, for example water vapor and/or further inert gases.

The processes are typically carried out continuously.

It is important that no liquid phase is formed during the reaction in the wall reactor, i.e. on the catalyst. This increases the operating life of the catalyst and reduces the need for regeneration.

In addition, other gases such as low-boiling organic solvents, ammonia or molecular oxygen can also be added to the feed gas mixture.

The olefin to be epoxidized can in principle be used in any ratio to the peroxidic component, preferably to the hydrogen peroxide.

In general, an increasing molar ratio of olefin to peroxidic component, preferably to H₂O₂, leads to increasing yields of epoxide. Preference is given to molar ratios of olefin to peroxidic compound in which the olefin is present in excess, preferably in the range from 1.1:1 to 30:1.

The gas-phase reactions are carried out at a temperature above 100° C., preferably at a temperature above 140° C. Preferred reaction temperatures are in the range from 140 to 700° C., in particular in the range from 140 to 250° C.

The gas-phase reactions are advantageously carried out in a pressure range from 0.05 to 4 MPa, preferably from 0.1 to 0.6 MPa.

The reaction mixture can be worked up in a manner known to those skilled in the art.

The process of the invention is simple to carry out and gives high space-time yields combined with high selectivity of the valuable oxidant.

Particular precautions for protection against explosions can be dispensed with in the particularly preferred microreactor.

The following examples illustrate the invention without restricting it.

All experiments were carried out in an apparatus comprising a vaporizer and a microreactor in which the hydraulic diameter was less than 1 mm and which comprised aluminum. Commercially available stabilized 50% strength by weight hydrogen peroxide solutions and various catalysts were used. Measurement and metering of the gas streams (propene, nitrogen) and the hydrogen peroxide solution were carried out using mass flow sensors from Bronkhorst.

A 50% strength by weight hydrogen peroxide solution and a gas mixture of propene and nitrogen which had been preheated to the vaporizer temperature were metered into the glass vaporizer (100° C.). The gas mixture leaving the vaporizer comprised 18 ml/min of H₂O₂, 53 ml/min of propene, 247 ml/min of N₂ and amounts of water and was reacted at various temperatures in the range from 100 to 180° C. in the microreactor. The reactor was for this purpose coated with 0.3 g of titanium silicalite-1 catalyst.

Contrary to expectations, a propylene oxide selectivity of the valuable oxidant which increased with increasing temperature was measured in the microreactor. The results are shown in the following table. When the reaction temperature was increased from 100 to 140° C., the selectivity increased by 100%.

| | | | | | |
|---|---|---|---|---|---|
| Reaction temperature (° C.) | 100 | 120 | 140 | 160 | 180 |
| PO selectivity of the | 15 | 27 | 32 | 33 | 37 |
| oxidant (%) | | | | | |

Krupper, Amal and Schüth have examined the influence of temperature on the heterogeneously catalyzed gas-phase epoxidation of propene by means of H₂O₂ over titanium silicalite-1 in a fixed-bed reactor made of glass (Europacat IV, 2003). The results are shown in the following table. As was actually to be expected, the PO selectivity of the H₂O₂ reacted decreased continually with increasing reaction temperature. When the reaction temperature was increased from 100° C. to 140° C., the selectivity decreased by 15%

| | | | | |
|---|---|---|---|---|
| Reaction temperature (° C.) | 100 | 120 | 140 | 150 |
| PO selectivity of the | 14 | 13 | 12 | 12 |
| oxidant (%) | | | | |

Accordingly, compared to the known state of the art, both increased propylene oxide selectivities of the oxidant and also increased space-time yields can be achieved with increasing temperature in an epoxidation in a microreactor. The effect cannot be achieved in a conventional fixed-bed reactor having hydraulic diameters of 1 cm. The critical hydraulic diameter for achieving the effect is accordingly below 1 cm.

1-22. (canceled)

23. A process for preparing an olefin oxide by heterogeneously catalyzed gas-phase epoxidation of an olefin by means of a peroxidic compound comprising the step of carrying out the gas-phase epoxidation at temperatures above 100° C. using a reactor which has at least one reaction space having at least one dimension of less than 10 mm, wherein the surface of the reaction space has a layer comprising aluminum oxide, zirconium oxide, tantalum oxide, silicon dioxide, tin oxide, glass and/or enamel and wherein the reaction space contains catalyst.

24. The process as claimed in claim 23, wherein a reactor in which the reaction space is coated or partly coated with catalyst is used.

25. The process as claimed in claim 23, wherein an olefin having from 2 to 6 carbon atoms is used as olefin and H₂O₂ is used as peroxidic compound.

26. The process as claimed in claim 23, wherein the reactor has a plurality of reaction spaces which run parallel to one another and each have at least one dimension of less than 1 mm.

27. The process as claimed in claim 26, wherein the gas-phase epoxidation is carried out in a microreactor which has a plurality of spaces which are arranged vertically or horizontally in parallel and each have at least one inlet and one outlet, with the spaces being formed by stacked plates or layers and part of the spaces representing reaction spaces and the other part of the spaces representing heat transport spaces and the inlets into the reaction spaces being connected to at least two distributor units and the outlets from the reaction spaces being connected to at least one collector unit and the heat transport between reaction spaces and heat transport spaces occurring through at least one common wall which is formed by a common plate.

28. The process as claimed in claim 27, wherein the microreactor has spacer elements in all spaces, contains catalyst material applied to at least part of the interior walls of the reaction spaces, has a hydraulic diameter defined as the ratio of four times the area to the circumference of the free flow cross section in the reaction spaces of less than 4000 μm and has a ratio of the vertically smallest distance between adjacent spacer elements to the slit height of the reaction space after coating with catalyst of less than 800 and greater than or equal to 10.

29. The process as claimed in claim 23, wherein a compound of an element of transition groups 4 to 6 of the Periodic Table and/or of arsenic or selenium and/or a molecular sieve is used as catalyst.

30. The process as claimed in claim 29, wherein a titanium-containing zeolite, in particular titanium silicalite-1 (TS-1) having an TiO₂ content in the range from 2 to 4%, is used as catalyst.

31. The process as claimed in claim 23, wherein a metal-organic compound, in particular an iron- or titanium-organic compound, is used as catalyst.

32. The process as claimed in claim 29, wherein an oxidic compound of vanadium or a molybdenum or tungsten compound selected from the group consisting of oxides, acids, molybdates, tungstates, molybdenum- or tungsten-containing homopolyacids or heteropolyacids and H₂O₂ adducts of these classes is used as catalyst.

33. The process as claimed in claim 23, wherein catalysts whose active component has been applied to a porous support are used.

34. The process as claimed in claim 23, wherein the catalyst is present together with a binder which is inert in respect of the epoxidation reaction on the surface of the reaction space.

35. The process as claimed in claim 34, wherein the inert binder consists essentially of aluminum oxide, silicon oxide or silicates.

36. The process as claimed in claim 23, wherein the gas-phase epoxidation is carried out at temperatures of from 140 to 700° C., preferably from 140 to 250° C.

37. The process as claimed in claim 23, wherein the gas mixture comprising olefin and peroxidic compound is contacted at a pressure in the range from 0.05 to 4 MPa.

38. The process as claimed in claim 23, wherein the gas mixture comprising olefin and peroxidic compound is used in a molar ratio of greater than 1:1, preferably in the range from 1.1:1 to 30:1.

39. A process for preparing a peroxidic compound by heterogeneously catalyzed reaction in the gas phase comprising the step of carrying out the reaction by reaction of a precursor of the peroxidic compound with oxygen and/or an oxygen-containing compound to form the peroxidic compound at temperatures above 100° C. using a reactor which has at least one reaction space having at least one dimension of less than 10 mm, wherein the surface of the reaction space has a layer comprising aluminum oxide, zirconium oxide, tantalum oxide, silicon dioxide, tin oxide, glass and/or enamel and wherein the reaction space may contain catalyst.
